SEQUENCE LISTING (4 sequences)

SEQ ID NO: 1 - protein, 585 aa, Escherichia coli
MAQVINTNSLSLITQNNINKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLAKDGSMKIQVGANDGETITIDLKKIDSDTLGLNGFNVNGKGTITNKAATVSDLTSAGAKLNTTTGLYDLKTENTLLTTDAAFDKLGNGDKVTVGGVDYTYNAKSGDFTTTKSTAGTGVDAAAQAADSASKRDALAATLHADVGKSVNGSYTTKDGTVSFETDSAGNITIGGSQAYVDDAGNLTTNNAGSAAKADMKALLKAASEGSDGASLTFNGTEYTIAKATPATTTPVAPLIPGGITYQATVSKDVVLSETKAAAATSSITFNSGVLSKTIGFTAGESSDAAKSYVDDKGGITNVADYTVSYSVNKDNGSVTVAGYASATDTNKDYAPAIGTAVNVNSAGKITTETTSAGSATTNPLAALDDAISSIDKFRSSLGAIQNRLDSAVTNLNNTTTNLSEAQSRIQDADYATEVSNMSKAQIIQQAGNSVLAKANQVPQQVLSLLQG

SEQ ID NO: 2 - protein, 495 aa, Salmonella typhimurium [sequence omitted]

SEQ ID NO: 3 - protein, 494 aa, Salmonella typhimurium
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 4 - protein, 4 aa, Artificial
  Synthetic peptide linker
GAAG

(19) United States
(12) Patent Application Publication
(10) Pub. No.: US 2017/0354709 A1
(43) Pub. Date: Dec. 14, 2017

(54) METHODS AND PHARMACEUTICAL COMPOSITIONS FOR THE TREATMENT OF ACUTE EXACERBATIONS OF CHRONIC OBSTRUCTIVE PULMONARY DISEASE

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); INSTITUT PASTEUR DE LILLE, Lille (FR); UNIVERSITÉ DE LILLE 1 SCIENCES ET TECHNOLOGIES, Villeneuve d'Ascq (FR); UNIVERSITÉ DE DROIT ET DE LA SANTÉ DE LILLE 2, Lille (FR)

(72) Inventors: Philippe GOSSET, Lille Cedex (FR); Muriel PICHAVANT, Lille Cedex (FR); Jean-Claude SIRARD, Lille Cedex (FR); Bachirou KONE, Lille Cedex (FR); Magdiel PEREZ-CRUZ, Lille Cedex (FR)

(21) Appl. No.: 15/539,380

(22) PCT Filed: Dec. 23, 2015
(86) PCT No.: PCT/EP2015/081111

§ 371 Date: Jun. 23, 2017

(30) Foreign Application Priority Data
Dec. 24, 2014 (EP) 14307184.3

(51) Int. Cl. A61K 38/16; A61K 45/06

(52) U.S. Cl. CPC A61K 38/164; A61K 45/06

(57) ABSTRACT

The present invention relates to methods and pharmaceutical compositions for the treatment of acute exacerbation of chronic obstructive pulmonary disease. In particular, the present invention relates to a method of treating acute exacerbation of chronic obstructive pulmonary disease in a subject in need thereof comprising administering the subject with a therapeutically effective amount of a flagellin polypeptide.

The present invention relates to methods and pharmaceutical compositions for the treatment of acute exacerbation of chronic obstructive pulmonary disease.

### BACKGROUND OF THE INVENTION

Chronic obstructive pulmonary disease (COPD) represents a severe and increasing global health problem. By 2020, COPD will have increased from 6th (as it is currently) to the 3rd most common cause of death worldwide. In the United States, COPD is believed to account for up to 120,000 deaths per year. The clinical course of COPD is characterized by chronic disability, with intermittent, acute exacerbations which may be triggered by a variety of stimuli including exposure to pathogens, inhaled irritants (e.g., cigarette smoke), allergens, or pollutants. “Acute exacerbation” refers to worsening of a subject's COPD symptoms from his or her usual state that is beyond normal day-to-day variations, and is acute in onset. Acute exacerbations of COPD greatly affect the health and quality of life of subjects with COPD. Acute exacerbation of COPD is a key driver of the associated substantial socioeconomic costs of the disease. Multiple studies have also shown that prior exacerbation is an independent risk factor for future hospitalization for COPD. In conclusion, exacerbations of COPD are of major importance in terms of their prolonged detrimental effect on subjects, the acceleration in disease progression and the high healthcare costs. However up to now there is no method for the treatment of acute exacerbation of COPD.

Modulating immunity by the activity of innate receptors is an emerging concept to elicit protective responses against infections. The rationale is to promote innate responses that greatly exceed in magnitude, quality and dynamic the innate response triggered by the pathogen itself. The effectiveness of TLR agonists for therapeutic treatment of infectious diseases has been demonstrated in several animal models, including models of respiratory tract infections. TLR5 senses bacterial flagellins that are the main constituent of flagella. Various cells of the pulmonary tract including the epithelial cells express TLR5 but the modulation of the TLR5 signalling pathway has not yet been investigated for the treatment of acute exacerbation of chronic obstructive pulmonary disease.

### SUMMARY OF THE INVENTION

The present invention relates to methods and pharmaceutical compositions for the treatment of acute exacerbation of chronic obstructive pulmonary disease. In particular, the present invention is defined by the claims.

### DETAILED DESCRIPTION OF THE INVENTION

The present invention relates to a method of treating acute exacerbation of chronic obstructive pulmonary disease in a subject in need thereof comprising administering the subject with a therapeutically effective amount of a flagellin polypeptide.

As used herein the term “acute exacerbation” has its general meaning in the art and refers to worsening of a subject's COPD symptoms from his or her usual state that is beyond normal day-to-day variations, and is acute in onset. Typically, the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, and said method comprises reducing the frequency, severity or duration of one or more of said symptoms. Acute exacerbation may have various etiologies, but typically may be caused by viral infections, bacterial infections, or air pollution. For example, approximately 50% of acute exacerbations are due primarily to the bacteria Streptococcus pneumoniae, Haemophilus influenzae, and Moraxella catarrhalis (all of them causing pneumonia). Viral pathogens associated with acute exacerbations in subjects with COPD include rhinoviruses, influenza, parainfluenza, coronavirus, adenovirus, and respiratory syncytial virus.

In some embodiments, the acute exacerbation of COPD is caused by a bacterial infection. In some embodiments, the acute exacerbation of COPD is caused by a viral infection. In some embodiments, the acute exacerbation of COPD is caused by air pollution.

In some embodiments, the subject experienced an acute exacerbation of COPD or is at risk of experiencing an acute exacerbation of COPD. In some embodiments, the subject has experienced at least one acute exacerbation of COPD in the past 24 months. In one particular embodiment, the subject has experienced at least one acute exacerbation of COPD in the past 12 months. In some embodiments, subject is a frequent exacerbator. As used herein the term “frequent exacerbator” refers to a subject who suffers from or is undergoing treatment for COPD and who experiences at least 2, and more typically 3 or more, acute exacerbations during a 12 month period.

In some embodiments, “treating” refers to treating an acute exacerbation of COPD, reducing the frequency, duration or severity of an acute exacerbation of COPD, treating one or more symptoms of acute exacerbation of COPD, reducing the frequency, duration or severity of one or more symptoms of an acute exacerbation of COPD, preventing the incidence of acute exacerbation of COPD, or preventing the incidence of one or more symptoms of acute exacerbation of COPD, in a human. The reduction in frequency, duration or severity is relative to the frequency, duration or seventy of an acute exacerbation or symptom in the same human not undergoing treatment according to the methods of the present invention. A reduction in frequency, duration or severity of acute exacerbation or one or more symptoms of acute exacerbation may be measured by clinical observation by an ordinarily skilled clinician with experience of treating COPD subjects or by subjective self evaluations by the subject undergoing treatment. Clinical observations by an ordinarily skilled clinician may include objective measures of lung function, as well as the frequency with which intervention is required to maintain the subject in his or her most stable condition, and the frequency of hospital admission and length of hospital stay required to maintain the subject in his or her most stable condition. Typically, subjective self evaluations by a subject are collected using industry-recognized and/or FDA-recognized subject reported outcome (PRO) tools. Such tools may allow the subject to evaluate specific symptoms or other subjective measures of quality of life. An example of one subject reported outcome tool is Exacerbations from Pulmonary Disease Tool (EXACT-PRO), which is currently being developed for evaluating clinical response in acute bacterial exacerbations by United BioSource Corporation along with a consortium of pharmaceutical industry sponsors in consultation with the FDA.

In some embodiments, the treatment is a prophylactic treatment. As used herein, the term “prophylactic treatment” refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

As used herein, the term “flagellin” is intended to mean the flagellin contained in a variety of Gram-positive or Gram-negative bacterial species. Non-limiting sources of flagellins include but are not limited to Escherichia, e.g., E. coli, Enterobacter, Erwinia, Klebsiella, Proteus, Salmonella, e.g., Salmonella enterica serovar Typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting. The amino acid sequences and nucleotide sequences of flagellins are publically available in the NCBI Genbank, see for example Accession Nos. AAL20871, NP_310689, BAB58984, AAO85383, AAA27090, NP_461698, AAK58560, YP_001217666, YP_002151351, YP_001250079, AAA99807, CAL35450, AAN74969, and BAC44986. The flagellin sequences from these and other species are intended to be encompassed by the term flagellin as used herein. Therefore, the sequence differences between species are included within the meaning of the term.

The term “flagellin polypeptide” is intended to a flagellin or a fragment thereof that retains the ability to bind and activate TLR5. As used herein the term “toll-like receptor 5” or “TLR5” has its general meaning in the art and is intended to mean a toll-like receptor 5 of any species, but preferably a human toll-like receptor 5. Upon activation, a TLR5 induces a cellular response by transducing an intracellular signal that is propagated through a series of signaling molecules from the cell surface to the nucleus. Typically, the intracellular domain of TLR5 recruits the adaptor protein, MyD88, which recruits the serine/threonine kinases IRAK (IRAK-1 and IRAK-4). IRAKs form a complex with TRAF6, which then interacts with various molecules that participate in transducing the TLR signal. These molecules and other TLR5 signal transduction pathway components stimulate the activity of transcription factors, such as fos, jun and NF-kB, and the corresponding induction of gene products of fos-, jun- and NF-kB-regulated genes, such as, for example, IL-6, TNF-alpha, CXCL1, CXCL2 and CCL20. Typically, the flagellin polypeptide of the present invention comprises the domains of flagellin involved in TLR5 signaling. The term “domain of flagellin” includes naturally occurring domain of flagellin and function conservative variants thereof “Function conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence identity between any two proteins of similar function may vary and may be, for example, from 70% to 99%. Thus a “function-conservative variant” also includes a polypeptide which has at least 70% amino acid identity with the native sequence of flagellin or fragment thereof. According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99, or 100% of identity with the second amino acid sequence. In the same manner a first amino acid sequence having at least 90% of identity with a second amino acid sequence means that the first sequence has 90; 91; 92; 93; 94; 95; 96; 97; 98; or 99, or 100% of identity with the second amino acid sequence. Amino acid sequence identity is preferably determined using a suitable sequence alignment algorithm and default parameters, such as BLAST P (Karlin and Altschul, 1990). The domains of flagellin that are involved in TLR5 signaling are well known in the art, see for example Smith et al. (2003) Nat. Immunol. 4: 1247-1253 (e.g., amino acids 78-129, 135-173 and 394-444 of S. typhimurium flagellin or homologs or modified forms thereof).

Examples of flagellin polypeptides include but are not limited to those described in U.S. Pat. Nos. 6,585,980; 6,130,082; 5,888,810; 5,618,533; and 4,886,748; U.S. Patent Publication No. US 2003/0044429 A1; and in the International Patent Application Publications n^{o} WO 2008097016 and WO 2009156405 which are incorporated by reference. An exemplary E. coli O157:H7 flagellin is SEQ ID NO:1. An exemplary S. typhimurium flagellin is SEQ ID NO:2 or SEQ ID NO:3.

In some embodiments, amino acid sequences having at least 70% of identity with SEQ ID NO: 1 SEQ ID NO:2 or SEQ ID NO:3 can be used as flagellin polypeptides according to the invention. In some embodiments, amino acid sequences having at least 90% of identity with SEQ ID NO: 1 SEQ ID NO:2 or SEQ ID NO:3 can be used as flagellin polypeptides according to the invention. In some embodiments, amino acid sequences having at least 70% of identity with SEQ ID NO:3 can be used as flagellin polypeptides according to the invention provided that the residues 89-96 (i.e. the residues that are involved in TLR5 detection) are not mutated (i.e. not substituted or not deleted). In some embodiments, amino acid sequences having at least 90% of identity with SEQ ID NO: 1 SEQ ID NO:2 or SEQ ID NO:3 can be used as flagellin polypeptides according to the invention provided that the residues 89-96 (i.e. the residues that are involved in TLR5 detection) are not mutated (i.e. not substituted or not deleted).

In some embodiments, the present invention encompasses use of the flagellin recombinant polypeptides described in the International Patent Application n° WO 2009156405 which is incorporated by reference in its entirely.

In some embodiments, the flagellin polypeptide of the present invention comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain. In some embodiments, said N-terminal peptide is selected from the group consisting of the amino acid sequences 1-99, 1-137, 1-160 and 1-173 of SEQ ID NO:3. In some embodiments, said C-terminal peptide is selected from the group consisting of the amino acid sequences 401-494 and 406-494 of SEQ ID NO:3. In some embodiments, said N-terminal and C-terminal peptides consist of the amino acid sequences 1-173 and 401-494 of SEQ ID NO:3, respectively. In some embodiments, said N-terminal and C-terminal peptides consist of the amino acid sequences 1-160 and 406-494 of SEQ ID NO:3, respectively. In some embodiments, said N-terminal and C-terminal peptides consist of the amino acid sequences 1-137 and 406-494 of SEQ ID NO:3, respectively. In some embodiments, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence. In some embodiments, the asparagine amino acid residue located at position 488 of SEQ ID NO:3 is replaced by a serine. In some embodiments, the flagellin polypeptide as above described comprises an additional methionine residue at the N-terminal end.

The flagellin polypeptide of the present invention is produced by any method well known in the art. In some embodiments, the flagellin polypeptide of the present invention is typically recombinantly produced by recombinant cells that have been transfected with a nucleic acid that encodes its amino acid sequence and allows its effective production within the transfected cells. The nucleic acid sequence encoding the flagellin polypeptide of the invention, may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence if the sequence is to be secreted, an origin of replication, one or more marker genes, an enhancer element, a promoter, and a transcription termination sequence. Construction of suitable vectors containing one or more of these components employs standard ligation techniques that are known to the skilled artisan. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. An example of suitable selectable markers for mammalian cells are those that enable the identification of cells competent to take up the nucleic acid encoding the flagellin polypeptide of the invention such as DHFR or thymidine kinase. An appropriate host cell when wild-type DHFR is employed is the CHO cell line deficient in DHFR activity. Expression and cloning vectors usually contain a promoter operably linked to the nucleic acid sequence encoding the flagellin polypeptide to direct mRNA synthesis. Promoters recognized by a variety of potential host cells are well known. Promoters suitable for use with prokaryotic hosts include the beta-lactamase and lactose promoter systems, alkaline phosphatase, a tryptophan (trp) promoter system, and hybrid promoters such as the tac promoter. Promoters for use in bacterial systems also will contain a Shine-Dalgarno (S. D.) sequence operably linked to the DNA encoding the flagellin polypeptide of the invention. Host cells are transfected or transformed with expression or cloning vectors described herein for flagellin polypeptide production and cultured in conventional nutrient media modified as appropriate for inducing promoters, selecting transformants, or amplifying the genes encoding the desired sequences. The culture conditions, such as media, temperature, pH, and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991). Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325); and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published 12 Apr. 1989), Pseudomonas such as P. aeruginosa, and Streptomyces. These examples are illustrative rather than limiting. Strain SIN41 of Salmonella typhimurium (fliC fljB), is particularly interesting for the production of flagellin polypeptides of the invention, since these prokaryotic host cells do not secrete any flagellins (Proc Natl Acad Sci USA. 2001; 98:13722-7). However flagellins are secreted through specialized secretion system: the so called “Type III secretion system”. Interestingly, strain SIN41 produces all components of the type III secretion system required for optimal flagellin secretion. Cloning sequence coding new flagellin peptides under fliC promoter enables secretion in large amounts of the flagellin polypeptides of interest in strain SIN41. Strain W3110 is also interesting because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W31 10 strain 27C7 (ATCC 55,244), which has the complete genotype tonA ptr3 phoA E15 (argF-lac)169 degP ompT kan.sup.r; E. coli W31 10 strain 37D6, which has the complete genotype tona ptr3 phoA E15 (argF-lac)169 degP ompT rbs7 ilvG kan.sup.r; E. coli W31 10 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. The E. coli strains MG1655, MG1655 AfimA-H or MKS12, a fliD- and -f/m>A-/-/-deleted MG1655 strain are also interesting candidates for production of recombinant flagellins as secreted proteins (Nat Biotechnol. 2005; (4):475-81). Alternatively, in vitro methods of cloning, e.g., PCR or other nucleic acid polymerase reactions, are suitable. Flagellin polypeptide of the invention may be recovered from culture medium or from host cell lysates. If membrane-bound, it can be released from the membrane using a suitable detergent solution (e.g., TRITON-X™ 100) or by enzymatic cleavage. In some embodiments, the flagellin polypeptide is purified from the supernatant of recombinant S. Typhimurium SIN41 (fliC fljB), as disclosed in Nempont et al. (Nempont, C. C., D.; Rumbo, M.; Bompard, C.; Villeret, V.; Sirard, J. C. 2008. Deletion of flagellin's hypervariable region abrogates antibody-mediated neutralization and systemic activation of TLR5-dependent immunity. J Immunol 181:2036-2043.). In particular, Salmonella were grown in Luria-Bertani (LB) broth for 6-18 hours at 37° C. with agitation. The supernatant was filtered and saturated with 60% ammonium sulfate (Sigma Aldrich, USA). The precipitated materials were recovered by centrifugation, solubilization in 20 mM Tris/HCl pH7.5 and then dialysis. The proteins were further purified by successive rounds of hydroxyapatite, anion exchange, and size exclusion chromatography (Bio-Rad Laboratories, USA; GE Healthcare, Sweden). Lastly, the proteins were depleted of lipopolysaccharide (LPS) using a polymyxin B column (Pierce, USA). Using the Limulus assay (Associates of Cape Cod Inc., USA), the residual LPS concentration was determined to be less than 30 pg LPS per μg recombinant flagellin. Constructs encoding the flagellins may be generated by PCR and cloned into the expression vector pET22b+. The plasmids can be introduced in Escherichia coli BL21 (DE3) and protein production can be induced by adding IPTG 1 mM. After disruption on French press, the soluble fraction was depleted of lipopolysaccharide (LPS) using Triton X-114 extraction. If flagellins are found in the insoluble fraction after the French-press, inclusion bodies are denatured in presence of Urea 8M followed by dialysis and Triton X-114 extraction. The proteins can then be purified on anion exchange chromatography and gel filtration. Finally, proteins can be again depleted of LPS using a polymyxin B column (Pierce, USA).

In some aspects, the present invention also relates to a method of treating acute exacerbation of chronic obstructive pulmonary disease in a subject in need thereof comprising administering the subject with a therapeutically effective amount of a nucleic acid encoding for a flagellin polypeptide.

By a “therapeutically effective amount” is meant a sufficient amount of the flagellin polypeptide (or the nucleic acid encoding thereof) for the treatment of acute exacerbation of COPD at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (i.e. the flagellin polypeptide or the nucleic acid encoding thereof) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports.

Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In some embodiments, the pharmaceutical composition of the invention is administered topically (i.e. in the respiratory tract of the subject). Therefore, the compositions can be formulated in the form of a spray, aerosol, solution, emulsion, or other form well-known to one of skill in the art. If the method of the invention comprises intranasal administration of a composition, the composition can be formulated in an aerosol form, spray, mist or in the form of drops. In particular, the active ingredients for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

In some embodiments, the flagellin polypeptide is administered to the subject in combination with an anti-bacterial agent, such as antibiotics or antiviral agents.

In some embodiments, the antibiotic is selected from the group consisting of aminoglycosides, beta lactams, quinolones or fluoroquinolones, macrolides, sulfonamides, sulfamethaxozoles, tetracyclines, streptogramins, oxazolidinones (such as linezolid), rifamycins, glycopeptides, polymixins, lipo-peptide antibiotics.

Tetracyclines belong to a class that shares a four-membered ring structure composed of four fused 6-membered (hexacyclic) rings. The tetracyclines exhibit their activity by inhibiting the binding of the aminoacyl tRNA to the 30S ribosomal subunit in susceptible bacteria. Tetracyclines for use in the invention include chlortetracycline, demeclocycline, doxycycline, minocycline, oxytetracycline, chlortetracycline, methacycline, mecocycline, tigecycline, limecycline, and tetracycline. The tetracyclines are effective against many known organisms including a-hemolytic streptococci, nonhemolytic streptococci, gram negative bacilli, rickettsiae, spirochetes, Mycoplasma, and Chlamydia.

Aminoglycosides are compounds derived from species of Streptomyces or Micomonospora bacteria and are primarily used to treat infections caused by gram-negative bacteria. Drugs belonging to this class all possess the same basic chemical structure, i.e., a central hexose or diaminohexose molecule to which two or more amino sugars are attached by a glycosidic bond. The aminoglycosides are bactericidal antibiotics that bind to the 30S ribosome and inhibit bacterial protein synthesis. They are active primarily against aerobic gram-negative bacilli and staphylococci. Aminoglycoside antibiotics for use in the invention include amikacin (Amikin®), gentamicin (Garamycin®), kanamycin (Kantrex®), neomycin (Mycifradin®), netilmicin (Netromycin®), paromomycin (Humatin®), streptomycin, and tobramycin (TOBI Solution®, TobraDex®).

Macrolides are a group of polyketide antibiotic drugs whose activity stems from the presence of a macrolide ring (a large 14-, 15-, or 16-membered lactone ring) to which one or more deoxy sugars, usually cladinose and desosamine, are attached. Macrolides are primarily bacteriostatic and bind to the 505 subunit of the ribosome, thereby inhibiting bacterial synthesis. Macrolides are active against aerobic and anaerobic gram positive cocci (with the exception of enterococci) and against gram-negative anaerobes. Macrolides for use in the invention include azithromycin (Zithromax®), clarithromycin (Biaxin®), dirithromycin (Dynabac®), erythromycin, clindamycin, josamycin, roxithromycin and lincomycin.

Ketolides belong to a class of semi-synthetic 14-membered ring macrolides in which the erythromycin macrolactone ring structure and the D-desosamine sugar attached at position 5 are retained, however, replacing the L-cladinose5 moiety and hydroxyl group at position 3 is a3-keto functional group. The ketolides bind to the 23S rRNA, and their mechanism of action is similar to that of macrolides (Zhanel, G. G.,et al.,Drugs, 2001; 61(4):443-98).The ketolides exhibit good activity against gram-positive aerobes and some gram-negative aerobes, and possess excellent activity against Streptococcus spp. including mefA and ermB-producing Streptococcus pneumoniae, and Haemophilus influenzae. Representative ketolides for use in the invention include telithromycin (formerly known as HMR-3647), HMR 3004, HMR 3647, cethromycin, EDP-420, and ABT-773.

Structurally, the quinolones possess a 1,4 dihydro-4-oxo-quinolinyl moiety bearing an essential carboxyl group at position 3. Functionally, the quinolones inhibit prokaryotic type II topoisomerases, namely DNA gyrase and, in a few cases, topoisomerase IV, through direct binding to the bacterial chromosome. Quinolones for use in the invention span first, second, third and fourth generation quinolones, including fluoroquinolones. Such compounds include nalidixic acid, cinoxacin, oxolinic acid, flumequine, pipemidic acid, rosoxacin, norfloxacin, lomefloxacin, ofloxacin, enrofloxacin, ciprofloxacin, enoxacin, amifloxacin, fleroxacin, gatifloxacin, gemifloxacin, clinafloxacin, sitafloxacin, pefloxacin, rufloxacin, sparfloxacin, temafloxacin, tosufloxacin, grepafloxacin, levofloxacin, moxifloxacin, and trovafloxacin. Additional quinolones suitable for use in the invention include those described in Hooper, D., and Rubinstein, E., “Quinolone Antimicrobial Agents, Vd Edition”, American Society of Microbiology Press, Washington D.C. (2004).

Drugs belonging to the sulfonamide class all possess a sulfonamide moiety, SO2NH2, or a substituted sulfonamide moiety, where one 15 of the hydrogens on the nitrogen is replaced by an organic substituent. Illustrative N-substituents include substituted or unsubstituted thiazole, pyrimidine, isoxazole, and other functional groups. Sulfonamide antibiotics all share a common structural feature, i.e., they are all benzene sulfonamides, meaning that the sulfonamide functionality is directly attached to a benzene ring. The structure of sulfonamide antibiotics is similar to p-aminobenzoic acid (PABA), a compound that is needed in bacteria as a substrate for the enzyme, dihydropteroate synthetase, for the synthesis of tetrahydrofolic acid. The sulfonamides function as antibiotics by interfering with the metabolic processes in bacteria that require PABA, thereby inhibiting bacterial growth and activity. Sulfonamide antibiotics for use in the invention include the following: mafenide, phtalylsulfathiazole, succinylsulfathiazole, sulfacetamide, sulfadiazine, sulfadoxine, sulfamazone, sulfamethazine, sulfamethoxazole, sulfametopirazine, sulfametoxypiridazine, sulfametrol, sulfamonomethoxine, sulfamylon, sulfanilamide, sulfaquinoxaline, sulfasalazine, sulfathiazole, sulfisoxazole, sulfisoxazole diolamine, and sulfaguanidine.

All members of beta-lactams possess a beta-lactam ring and a carboxyl group, resulting in similarities in both their pharmacokinetics and mechanism of action. The majority of clinically useful beta-lactams belong to either the penicillin group or the cephalosporin group, including cefamycins and oxacephems. The beta-lactams also include the carbapenems and monobactams. Generally speaking, beta-lactams inhibit bacterial cell wall synthesis. More specifically, these antibiotics cause ‘nicks’ in the peptidoglycan net of the cell wall that allow the bacterial protoplasm to flow from its protective net into the surrounding hypotonic medium. Fluid then accumulates in the naked protoplast (a cell devoid of its wall), and it eventually bursts, leading to death of the organism. Mechanistically, beta-lactams act by inhibiting D-alanyl-D-alanine transpeptidase activity by forming stable esters with the carboxyl of the open lactam ring attached to the hydroxyl group of the enzyme target site. Beta-lactams are extremely effective and typically are of low toxicity. As a group, these drugs are active against many gram-positive, gram-negative and anaerobic organisms. Drugs falling into this category include 2-(3-alanyl)clavam, 2-hydroxymethylclavam, 7-methoxycephalosporin, epi-thienamycin, acetyl-thienamycin, amoxicillin, apalcillin, aspoxicillin, azidocillin, azlocillin, aztreonam, bacampicillin, blapenem, carbenicillin, carfecillin, carindacillin, carpetimycin A and B, cefacetril, cefaclor, cefadroxil, cefalexin, cefaloglycin, cefaloridine, cefalotin, cefamandole, cefapirin, cefatrizine, cefazedone, cefazolin, cefbuperazone, cefcapene, cefdinir, cefditoren, cefepime, cefetamet, cefixime, cefinenoxime, cefinetazole, cefminox, cefmolexin, cefodizime, cefonicid, cefoperazone, ceforamide, cefoselis, cefotaxime, cefotetan, cefotiam, cefoxitin, cefozopran, cefpiramide, cefpirome, cefpodoxime, cefprozil, cefquinome, cefradine, cefroxadine, cefsulodin, ceftazidime, cefteram, ceftezole, ceftibuten, ceftizoxime, ceftriaxone, cefuroxime, cephalosporin C, cephamycin A, cephamycin C, cephalothin, chitinovorin A, chitinovorin B, chitinovorin C, ciclacillin, clometocillin, cloxacillin, cycloserine, deoxy pluracidomycin B and C, dicloxacillin, dihydro pluracidomycin C, epicillin, epithienamycin D, E, and F, ertapenem, faropenem, flomoxef, flucloxacillin, hetacillin, imipenem, lenampicillin, loracarbef, mecillinam, meropenem, metampicillin, meticillin (also referred to as methicillin), mezlocillin, moxalactam, nafcillin, northienamycin, oxacillin, panipenem, penamecillin, penicillin G, N, and V, phenethicillin, piperacillin, povampicillin, pivcefalexin, povmecillinam, pivmecillinam, pluracidomycin B, C, and D, propicillin, sarmoxicillin, sulbactam, sultamicillin, talampicillin, temocillin, terconazole, thienamycin, andticarcillin.

Over 400 natural antimicrobial peptides have been isolated and characterized. Based on chemical structure, these peptides may be classified into two main groups: linear and cyclic (R. E. Hancock et al, Adv. Microb. Physiol., 1995, 37: 135-137; H. Kleinkauf et al., Criti. Rev. Biotechnol., 198, 8: 1-32; D. Perlman and M. Bodansky, Annu. Rev. Biochem., 1971, 40: 449-464). The mode of action for the majority of these peptides (both linear and cyclic) is believed to involve membrane disruption, leading to cell leakage (A. Mor, Drug Develop. Res., 2000, 50: 440-447). The linear peptides, such as magainins and melitting, exist mainly as a-helical amphipathic structures (containing segregated hydrophobic and hydrophilic moieties), or as β-helices as found in gramicidin A (GA). Cyclic peptides, which mainly adopt an amphipatic β-sheet structures can be further divided into two subgroups: those containing disulfide bonds, such as tachyplesin, and those that do not, such as gramicidin S (D. Audreu and L. Rivas, Biopolymers, 1998, 47: 415-433). Peptide antibiotics also fall into two classes: non-ribosomally synthesized peptides, such as the gramicicins, polymyxins, bacitracins, glycopeptides, etc., and ribosomally synthesized (natural) peptides. The former are often drastically modified and are largely produced by bacteria, whereas the latter are produced by all species of life (including bacteria) as a major component of the natural host defense molecules of these species. In certain embodiments, the peptide antibiotic is a lipopeptide antibiotic such as colistin, daptomycin, surfactin, friulimicin, aculeacin A, iturin A, and tsushimycin. Colistin (also called Colimycin) is a polymixin antibiotic discovered more than 50 years ago. It is a cyclic lipopeptide antibiotic which penetrates the cell wall of Gram negative bacteria by self-induced mechanism by chelating divalent ions. Colistin destabilizes the wall and can insinuate into it. Colistin basically perforates the cell wall, causing distortion of this structure and the release of intracellular constituents. Increasing multidrug resistance in Gram-negative bacteria, in particular Pseudomonas aeruginosa, Acinetobacter baumannii, and Klebsiella pneumoniae, presents a critical problem. Limited therapeutic options have forced infectious disease clinicians and microbiologists to reappraise the clinical application of Colistin. Colistin is associated with neurotoxicity and nephrotoxicity. Dosage regimen and novel formulation may be an answer to address the toxicity issue.

Combination treatment may also include respiratory stimulants. Corticosteroids may be beneficial in acute exacerbations of COPD. Examples of corticosteroids that can be used in combination with the agonist of the present invention are prednisolone, methylprednisolone, dexamethasone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate. Particularly preferred corticosteroids under the present invention are: dexamethasone, budesonide, beclomethasone, triamcinolone, mometasone, ciclesonide, fluticasone, flunisolide, dexamethasone sodium phosphate and esters thereof as well as 6α,9α-difluoro-17α-[(2-furanylcarbonyl)oxy]-11β-hydroxy-16α-methyl-3-oxoandrosta-1,4-diene-17β-carbothioic acid (S)-fluoromethyl ester. Still more preferred corticosteroids under the present invention are: budesonide, beclomethasone dipropionate, mometasone furoate, ciclesonide, triamcinolone, triamcinolone acetonide, triamcinolone hexaacetonide and fluticasone propionate optionally in the form of their racemates, their enantiomers, their diastereomers and mixtures thereof, and optionally their pharmacologically-compatible acid addition salts. Even more preferred are budesonide, beclomethasone dipropionate, mometasone furoate, ciclesonide and fluticasone propionate. The most preferred corticosteroids of the present invention are budesonide and beclomethasone dipropionate.

Bronchodilator dosages may be increased during acute exacerbations to decrease acute bronchospasm. Examples of bronchodilators include but are not limited to β2-agonists (e.g. salbutamol, bitolterol mesylate, formoterol, isoproterenol, levalbuterol, metaproterenol, salmeterol, terbutaline, and fenoterol), anticholinergic (e.g. tiotropium or ipratropium), methylxanthined, and phosphodiesterase inhibitors.

In some embodiments, the flagellin polypeptide of the present invention is administered to the subject in combination with a vaccine which contains an antigen or antigenic composition capable of eliciting an immune response against a virus or a bacterium. Typically, the vaccine composition is used to eliciting an immune response against at least one bacterium selected from the group consisting of Streptococcus pneumoniae, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis. In particular, the vaccine composition is directed against Streptococcus pneumonia or Haemophilus influenza. More particularly, the vaccine composition is directed against Non-typeable Haemophilus influenzae (NTHi). Typically, vaccine composition typically contains whole killed or inactivated (eg., attenuated) bacteria isolate(s). However, soluble or particulate antigen comprising or consisting of outer cell membrane and/or surface antigens can be suitable as well, or instead of, whole killed organisms. In one or more embodiments, the outer cellular membrane fraction or membrane protein(s) of the selected isolate(s) is used. For instance, NTHi OMP P6 is a highly conserved 16-kDa lipoprotein (Nelson, 1988) which is a target of human bactericidal antibody and induces protection both in humans and in animal models. In chronic pulmonary obstructive disease (COPD), OMP P6 has been shown to evoke a lymphocyte proliferative response that is associated with relative protection from NTHi infection (Abe, 2002). Accordingly, OMP P6 or any other suitable outer membrane NTHi proteins, polypeptides (eg., P2, P4 and P26) or antigenic fragments of such proteins or polypeptides can find application for a NTHi vaccine. Soluble and/or particulate antigen can be prepared by disrupting killed or viable selected isolate(s). A fraction for use in the vaccine can then be prepared by centrifugation, filtration and/or other appropriate techniques known in the art. Any method which achieves the required level of cellular disruption can be employed including sonication or dissolution utilizing appropriate surfactants and agitation, and combination of such techniques. When sonication is employed, the isolate can be subjected to a number of sonication steps in order to obtain the required degree of cellular disruption or generation of soluble and/or particulate matter of a specific size or size range. In some embodiments, the vaccine composition comprises an adjuvant, in a particular TLR agonist. In one embodiment, the TLR agonist is selected from the group consisting of TLR1, TLR2, TLR3, TLR4, TLR5, TLR6, TLR7, TLR8, TLR9, TLR10, TLR11, TLR12, or TLR13 agonists.

In certain embodiments, oxygen requirements may increase and supplemental oxygen may be provided.

The invention will be further illustrated by the following figures and examples. However, these examples and figures should not be interpreted in any way as limiting the scope of the present invention.

### FIGURES

FIG. 1—Alterations of IL-17 and IL-22 production in response to S. pneumoniae and not typeable heamophilus influenzae (NTHI) in COPD mice. Mice were chronically exposed to cigarette smoke over a period of 12 weeks and then intranasally challenged with 5×10⁴ CFU of Streptococcus pneumoniae (Sp) or not (Mock). IFNγ, IL-17 and IL-22 levels were evaluated in the BAL (A). Concentrations of 11-22 in the serum (B) and in supernatants from restimulated pulmonary cells (C) were measured 24h after Sp challenge. Mice chronically exposed to cigarette smoke over a period of 12 weeks were then intranasally challenged with 5×10⁷ CFU of NTHI or not (Mock). Results were expressed as mean±SEM (n>10 per group).

FIG. 2: Administration of flagellin decreased the bacterial load in S. pneumoniae infected COPD mice. Bacterial load was measured in the bronchoalveolar lavade (BAL) (A), the lung lysate (B) and the blood (C) of COPD (chronically exposed to cigarette smoke) and Air (controls) mice infected or not with 5×10⁴ cfu of streptococcus pneumoniae serotype 1 (SP1). A group of mice received 5 μg of flagellin (FLIC) by intraperitoneal route. Bacterial load was analyzed at day 1 and 3 after infection. Results were expressed as mean±SEM (n>6 per group).

FIG. 3: Administration of flagellin increased the IL-17 production at day 3 after infection by S. pneumoniae in COPD mice. Levels of IL17 were measured in the bronchoalveolar lavade (BAL) (A) and in supernatants of lung cells (B) from COPD (chronically exposed to cigarette smoke) and Air (controls) mice infected or not with 5×10⁴ cfu of streptococcus pneumoniae serotype 1 (SP1). A group of mice received 5 μg of flagellin (FLIC) by intraperitoneal route. Concentrations of IL-17 were analyzed by ELISA at day 1 and 3 after infection. Results were expressed as mean±SEM (n>6 per group).

FIG. 4: Administration of flagellin increased the IL-22 production at day 3 after infection by S. pneumoniae in COPD mice. Levels of IL-22 were measured in the bronchoalveolar lavade (BAL) (A) and in supernatants of lung cells (B) from COPD (chronically exposed to cigarette smoke) and Air (controls) mice infected or not with 5×104 cfu of streptococcus pneumoniae serotype 1 (SP1). A group of mice received 5 μg of flagellin (FLIC) by intraperitoneal route. Lung cells were cultivated for 48h in complete medium alone (unstimulated conditions) or were stimulated by fixed anti-CD3 antibody (Anti-CD3 conditions). Concentrations of IL-22 were analyzed by ELISA at day 1 and 3 after infection. Results were expressed as mean±SEM (n>6 per group).

FIG. 5: Administration of flagellin decreased the bacterial load at day 1 post infection by NTHI in COPD mice. Bacterial load was measured in the bronchoalveolar lavade (BAL) (A), the lung lysate (B) and the blood (C) of COPD (chronically exposed to cigarette smoke) and Air (controls) mice infected or not with 5×10⁷ cfu of not typable haemophilus influenza (NTHI). A group of mice received 5 μg of flagellin (FLIC) by intraperitoneal route. Bacterial load was analyzed at day 1 after infection. Results were expressed as mean±SEM (n>6 per group).

FIG. 6: Administration of flagellin increased the IL-22 production at day 1 after infection by NTHI in COPD mice. Levels of IL-22 were measured in the bronchoalveolar lavade (BAL) (A) and in supernatants of lung cells (B) from COPD (chronically exposed to cigarette smoke) and Air (controls) mice infected or not with 5×10⁷ cfu of not typable haemophilus influenza (NTHI). A group of mice received 5 μg of flagellin (FLIC) by intraperitoneal route. Lung cells were cultivated for 48h in complete medium alone (unstimulated conditions) or were stimulated by heat-killed NTHI (HK-NTHIconditions). Concentrations of IL-22 were analyzed by ELISA in lung cells collected at day 1 after infection. Results were expressed as mean±SEM (n>6 per group).

### EXAMPLE

Introduction:

Exacerbation episodes due to bacterial infection are a common feature during a wide variety of lung inflammatory disorders such as COPD. In patients with COPD, acute exacerbation is mostly associated with bacterial infections mostly due to Haemophilus influenzae and Streptococcus pneumoniae. Pathologically, exacerbations of COPD are characterized by enhanced oedema, airway and systemic inflammation, resulting in more airflow limitation and gas exchange defects. A growing body of evidence indicates that host innate immune defenses are broadly suppressed during COPD. We have developed appropriate models to better define mechanisms responsible for bacterial susceptibility during the exacerbation of COPD underlying on mice chronically exposed to cigarette smoke (CS) during 12 weeks (Pichavant M et al., Mucosal Immunol, 2014). For this, C57/BL6 mice chronically exposed to CS were infected by the local administration of sub-lethal doses of H. influenzae and S. pneumoniae. As reported in the first paragraph, these models have allowed to identify a strong defect in Th17 cytokines (IL-17A and IL-22) production after bacterial infection in COPD mice. Since TLR agonists have been identified as potent immuno-stimulators and as promoter of lung anti-bacterial defenses particularly against S. pneumoniae (ref JCS), we have then tested the ability of flagellin (a TLR5 agonist) to limit the development of bacterial infection and their consequences on lung inflammation in COPD mice

Material & Methods

Cigarette Smoke Exposure

C57/BL6 mice were exposed to CS generated from 5 cigarettes per day, 5 days a week, and up to 12 weeks using a smoke machine (Emka, Scireq, Canada). Air mice were housed in similar conditions and were only exposed to filtered air.

Bacterial Infection

Two strains of bacteria were used to exacerbate COPD: Streptococcus pneumoniae (Sp) serotype 1, and non typable Haemophilus influenza (NTHI). Bacteria stocks were kept frozen at −80° C. Bacteria were defrost just before the infection, and the number of cfu was determined on chocolate plates. Infection was performed by intranasal route (50 μl/mouse). Mice infected with S. pneumoniae were sacrificed at day 1 and day 3. Mice infected with NTHI were sacrificed at day 1 and day 2.

Administration of Flagellin

Purified recombinant flagellin were prepared in endotoxin-free conditions. Five μg of flagellin was injected by intraperitoneal route just before the infection in all the treated mice and two days after the infection in mice infected by S. pneumoniae. The controls were injected with the same volume of PBS (100 μl) (Mock).

Cytokine Quantification

Mouse IL-2, IL-17, IL-22 and IFN-gamma concentrations were measured in BAL, lung lysates and supernatants of lung cell culture by ELISA (R&D systems and e-Biosciences).

Reverse Transcriptase-Polymerase Chain Reaction (RT-PCR) Analysis

Quantitative RT-PCR was performed to quantify mRNA of interest (Table 1). Results were expressed as mean±SEM of folds (2^{-ΔΔCt}) of the gene expression using β-actin as a reference, and compared to controls (air) calculated for each experiment.

Results

IL-17 and IL-22 Response to Bacterial Infection is Altered in COPD Mice.

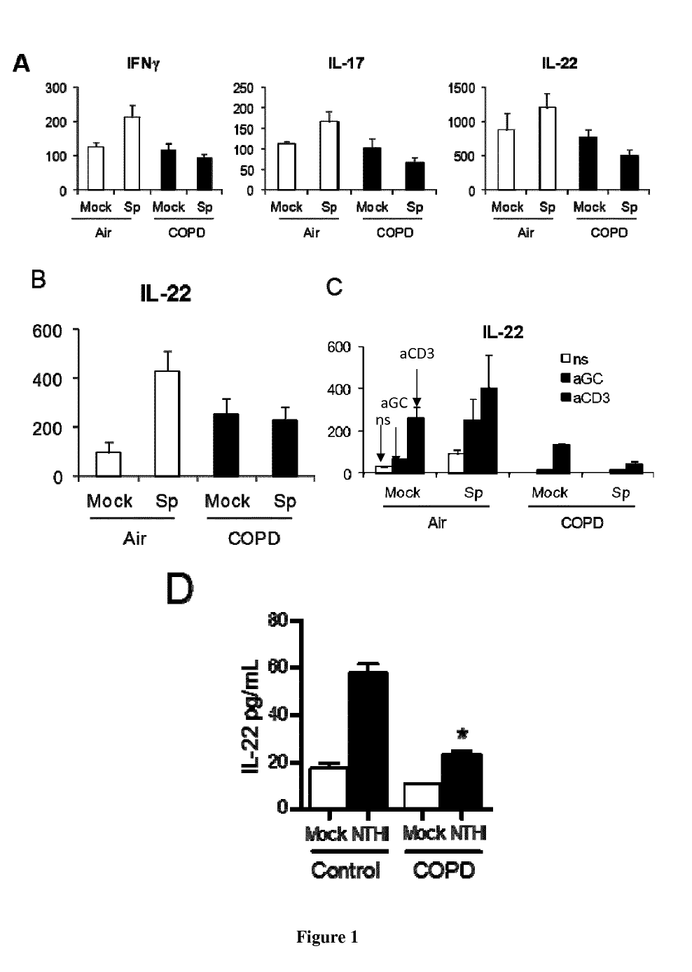

Infected COPD mice develop a strong lung infection with SP (associated with an increased inflammatory reaction) whereas naïve mice are able to clear the bacteria within 24 hours. This defect in bacterial clearance is associated with a lower production of both IL-17 and IL-22 in the BAL (FIG. 1A) and after restimulation of lung cells (FIG. 1C). IL-22 concentrations were also lower in the blood of infected COPD mice as compared with Air mice (FIG. 1B). The defect in IL-17 and IL-22 is related to a decreased percentage of NK, NKT cells as well as innate lymphoid cells (ILC) positive for these cytokines in the lung of infected COPD mice as compared to infected air-exposed mice. Moreover, the supplementation with recombinant IL-22 allows to accelerate the clearance of the bacteria and to limit the consequences of the infection by S. pneumoniae in COPD mice. In COPD mice, infection with NTHI is associated with a defect in the production of IL-22 as shown in the BAL fluid (FIG. 1D). This defect also involved the same cells as observed for SP (ILC, NK and NKT cells). Since the role of IL-22 is unknown during infection by NTHI, we observed that IL-22−/− mice reproduce a phenotype close to that of COPD mice (increased susceptibility, higher inflammatory response and lung remodeling). These data show that COPD mice are more susceptible to infection by H. influenzae and S. pneumoniae than control mice and a deficient production of IL-17 and/or IL-22 may favor the development of bacteria-induced COPD exacerbations.

Administration of Flagellin Limits the Consequences of Infection by S. pneumoniae in COPD Mice.

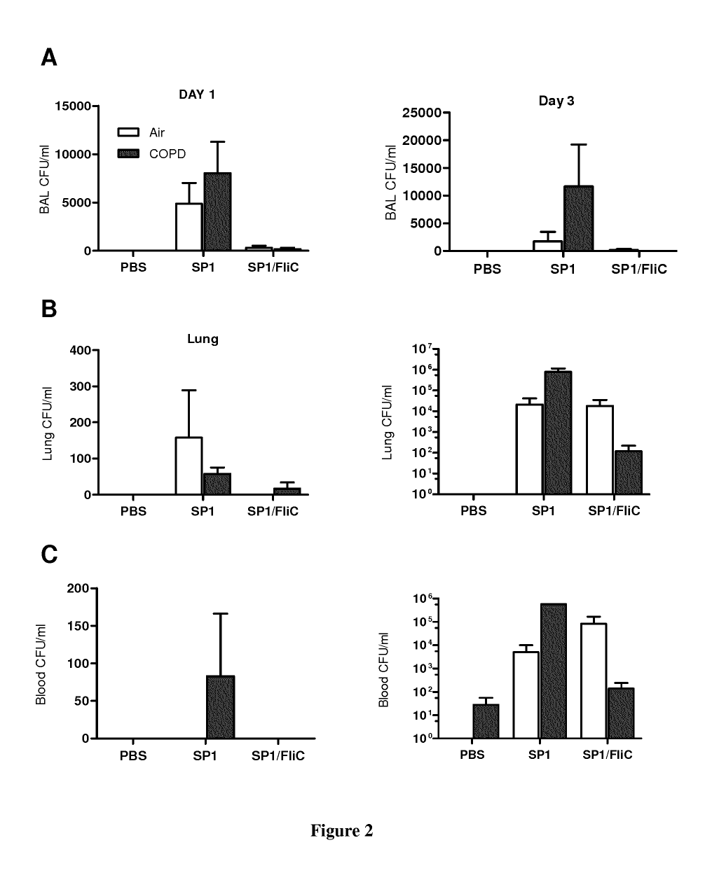

Administration of flagellin by intraperitoneal route markedly reduced the bacterial load in Air and COPD mice at day 1 and 3 after infection. The effect is more important in the BAL (FIG. 2A) and the blood (FIG. 2C) as compared with the lung (FIG. 2B).

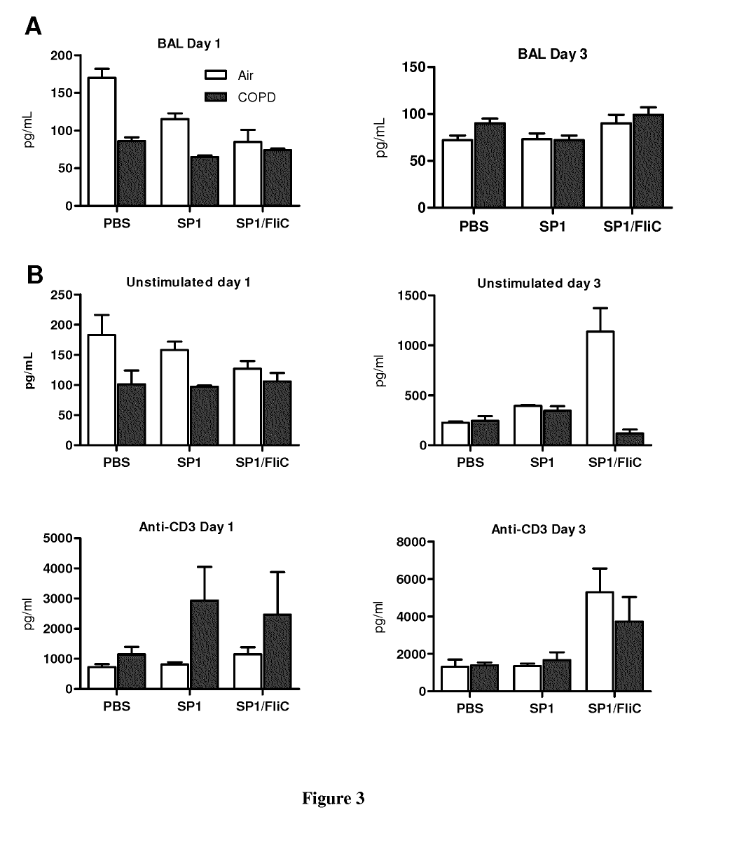

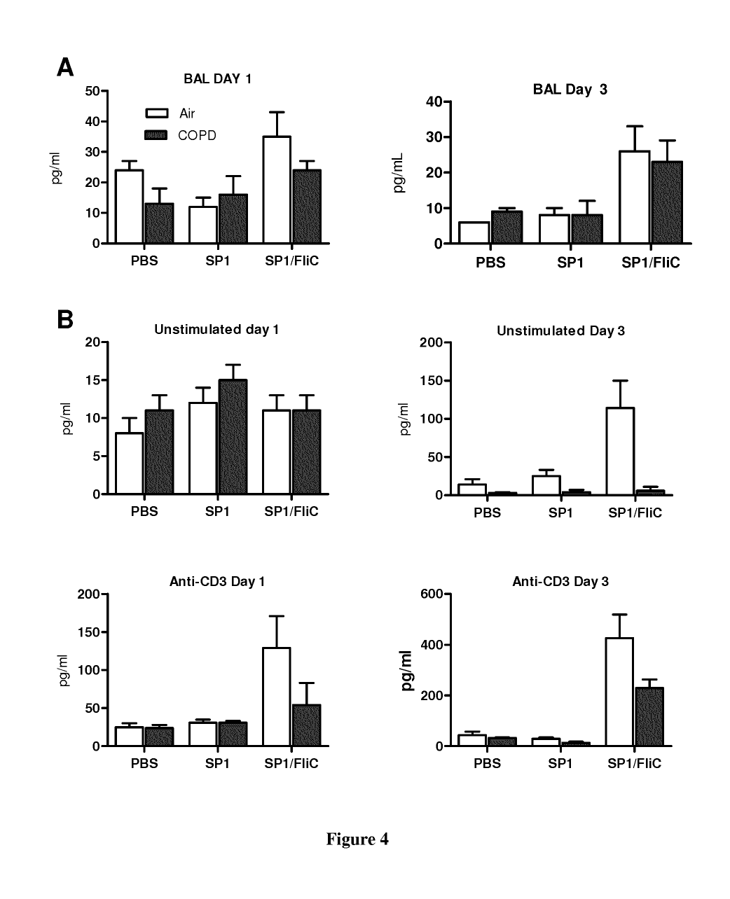

Analysis of the cytokine burst in the lung from infected animals revealed that administration of flagellin did not markedly increased the production of inflammatory cytokines (IL-1β, IL-6 and TNF-alpha) in infected Air and COPD mice. In contrast, treatment with flagellin enhanced the concentrations of IL-17 at day 3 in BAL fluids and in supernatants of anti-CD3 stimulated lung cells from both Air and COPD mice (FIG. 3). The same profile was observed for the secretion of IL-22 (FIG. 4) although the effect seems stronger for this cytokine than for IL-17. The modulation at day 3 of cytokine production was not associated with an increased number of neutrophils in the lung of infected mice receiving flagellin.

These data show that treatment with flagellin limits the bacterial susceptibility in COPD mice. This effect was associated with an increased production of IL-17 and IL-22 in the lung of infected COPD mice, both cytokines being involved in lung defenses against bacteria. Moreover, treatment with flagellin in infected COPD mice was not associated with a burst of inflammatory cytokines and an amplified recruitment of neutrophils which might be deleterious for the lung function.

Administration of Flagellin Limits the Consequences of Infection by NTHI in COPD Mice.

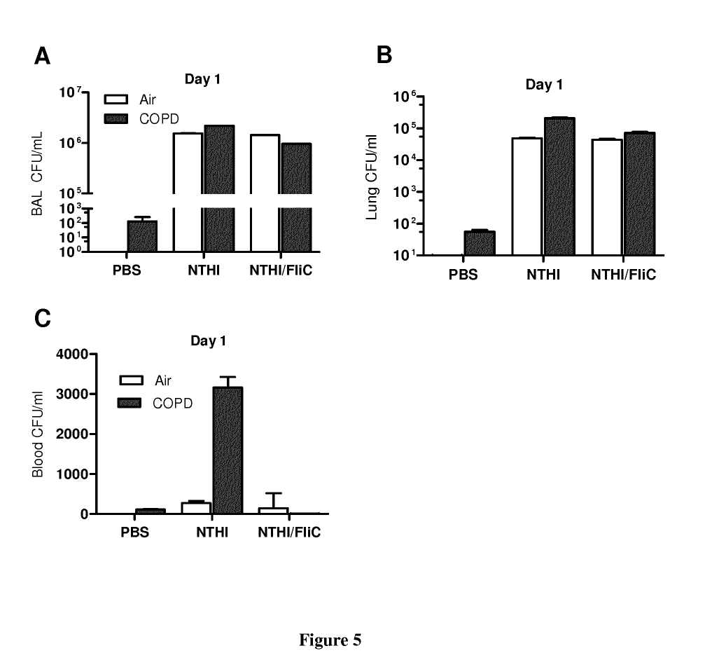

Similar experiments were reproduced in Air and COPD mice infected with NTHI. As showed with S. pneumoniae, administration of flagellin reduced the bacterial load in the BAL, the lung compartment and the blood (FIG. 5). The decrease reached 55 and 65% in the BAL and lung lysate, respectively, whereas it was nearly total in the blood.

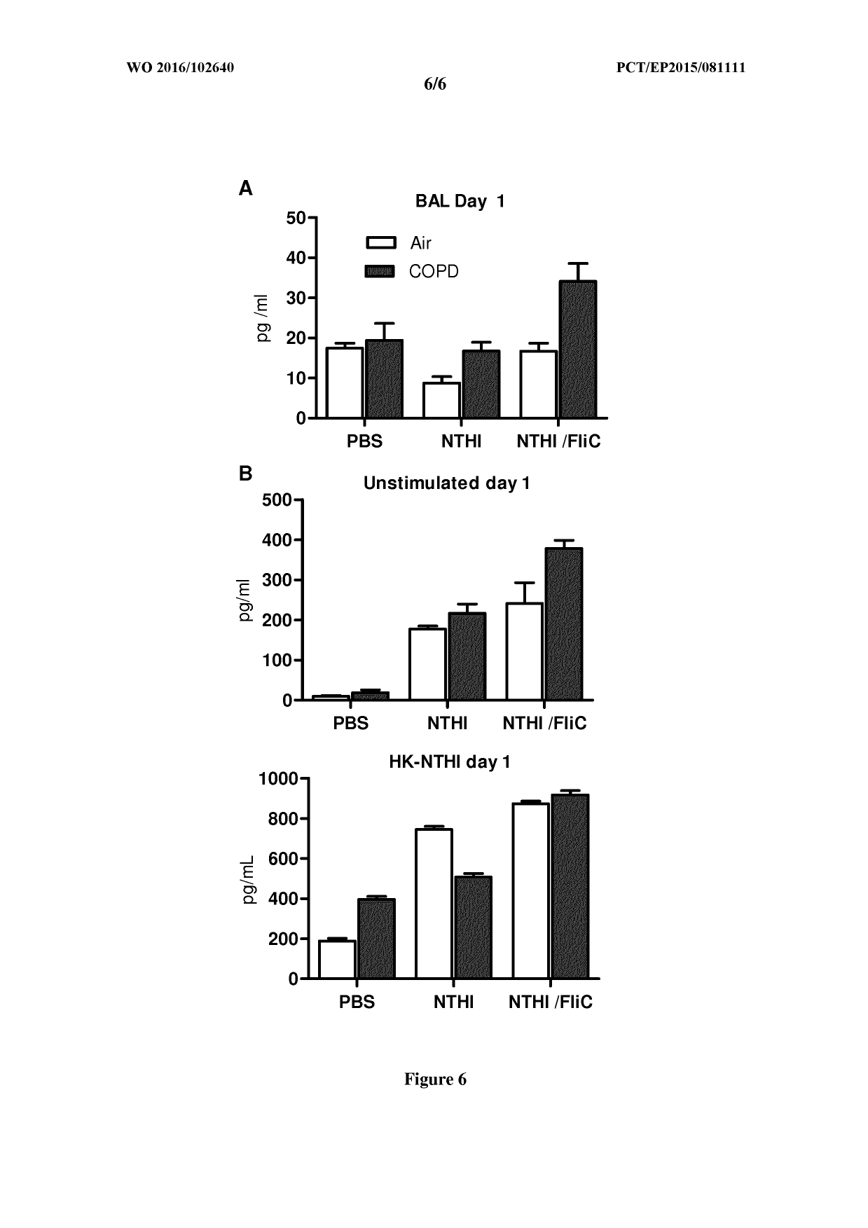

The effect of flagellin was measured on the cytokine production in NTHI-infected COPD mice. As previously reported with S. pneumoniae, treatment with flagellin did not increased the production of inflammatory cytokines (IL-1β, IL-6 and TNF-alpha) in the lung of infected Air and COPD mice. Moreover, the secretion of IL-17 was not modulated with this bacteria. In contrast, the synthesis of IL-22 in the BAL and the cultures of lung cells was markedly amplified in COPD mice at day 1 (FIG. 6). Similar findings were observed at day 2 post infection. The recruitment of neutrophils was not modulated by the treatment with flagellin.

Altogether, these data demonstrate that administration of flagellin is able to decrease the bacterial load in COPD mice infected with NTHI. Since we previously reported that IL-22 production is important for the control of this infection, we can suspect that the increase in IL-22 production might be implicated in the beneficial effect of flagellin in NTHI-infected COPD mice. This treatment did not obviously amplified the lung inflammation in NTHI-infected COPD mice.

### REFERENCES

Throughout this application, various references describe the state of the art to which this invention pertains. The disclosures of these references are hereby incorporated by reference into the present disclosure.

1. A method of treating acute exacerbation of chronic obstructive pulmonary disease in a subject in need thereof comprising administering the subject with a therapeutically effective amount of a flagellin polypeptide.

2. The method of claim 1 wherein the acute exacerbation of COPD is caused by a bacterial infection, by a viral infection or by air pollution.

3. The method of claim 2 wherein the bacterial infection is caused by Streptococcus pneumoniae, Haemophilus influenzae, or Moraxella catarrhalis.

4. The method of claim 1 wherein the subject experienced an acute exacerbation of COPD or is at risk of experiencing an acute exacerbation of COPD.

5. The method of claim 1 wherein the subject is a frequent exacerbator.

6. The method of claim 1 wherein the flagellin polypeptide has at least 70% of identity with SEQ ID NO:1, SEQ ID NO:2 or SEQ ID NO:3.

7. The method of claim 1 wherein the flagellin polypeptide comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain.

8. The method of claim 7 wherein the said N-terminal peptide is selected from the group consisting of the amino acid sequences 1-99, 1-137, 1-160 and 1-173 of SEQ ID NO:3.

9. The method of claim 7 wherein said C-terminal peptide is selected from the group consisting of the amino acid sequences 401-494 and 406-494 of SEQ ID NO:3.

10. The method of claim 7 wherein said N-terminal and C-terminal peptides consist of the amino acid sequences 1-173 and 401-494 of SEQ ID NO:3, respectively.

11. The method of claim 7 wherein said N-terminal and C-terminal peptides consist of the amino acid sequences 1-160 and 406-494 of SEQ ID NO:3, respectively.

12. The method of claim 7 wherein said N-terminal and C-terminal peptides consist of the amino acid sequences 1-137 and 406-494 of SEQ ID NO:3, respectively.

13. The method of claim 7 wherein said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence.

14. The method of claim 7 wherein the asparagine amino acid residue located at position 488 of SEQ ID NO:3 is replaced by a serine.

15. The method of claim 1 wherein the flagellin polypeptide is administered to the subject in combination with an antibiotic.
